(19) United States
(12) Patent Application Publication
(10) Pub. No.: US 2010/0284966 A1
(43) Pub. Date: Nov. 11, 2010

(54) Methods and Compositions for Managing Resistance of Hepatitis C Virus to Immunosuppressant

(76) Inventors: Robert T. Striker, Madison, WI (US); Dan S. Poole, Madison, WI (US); Fiona Fernandes, Madison, WI (US)

Correspondence Address: KENING LI;PINSENT MASONS LLP, c/o FOUNTAIN LAW GROUP, INC, 18201 Von Karman Avenue, Suite 960, Irvine, CA, 92612, US

(21) Appl. No.: 11/556,925

(22) Filed: Nov. 6, 2006

Related U.S. Application Data
(60) Provisional application No. 60/733,176, filed on Nov. 4, 2005.

(51) Int. Cl. A61K 38/21; C12Q 1/70; C07H 21/04; C12Q 1/68; A61P 31/12

(52) U.S. Cl. 424/85.4; 435/5; 536/23.1; 435/6

(57) ABSTRACT

Method for determining susceptibility of a hepatitis C virus (HCV) in a patient, especially one who received a liver transplant, to cyclosporine A (CsA), comprising determining the nucleic acid sequence of HCV, and comparing the viral nucleic acid sequence to that of a wild-type, CsA susceptible strain, wherein the existence of at least one mutation in the viral genome is indicative that the virus is resistant to CsA. Also provided are isolated polynucleotide molecules comprising one or more mutations, an array of such molecules, a kit comprising at least one isolated polynucleotide. A method of monitoring the development CsA resistance in a HCV patient, and managing treatment regimens accordingly is further provided. The present invention further provides CsA resistant HCV replicons and method of using them for anti-viral drug screening.

### BACKGROUND OF THE INVENTION

Hepatitis C viral (HCV) was first characterized in 1989 (Choo et al., 1989, Science 244: 359-362), although its existence had been suspected for many years as the elusive cause of a liver disease referred to as non A-non B hepatitis (“NANBH”), with flu-like symptoms and occurring in many patients years after they receive blood transfusion. HCV is a single-stranded, plus-sense RNA virus of Flaviviridae, which includes viruses that cause bovine diarrhea, hog cholera, yellow fever, and tick-borne encephalitis. The HCV genome is approximately 9.5 kb in size, and is characterized by a unique open reading frame encoding a single poly-protein.

It is estimated that HCV infects about 170 million people worldwide, more than four times those infected with human immunodeficiency virus (“HIV”), and the number of HCV-associated deaths may eventually overtake deaths caused by AIDS (Cohen, 1999, Science 285: 26-30). The Center for Disease Control (CDC) has calculated that 1.8 percent of the U.S. population may be infected with HCV.

HCV infection is now known to be the leading cause of liver failure in the United States. Approximately 60% of HCV patients develop chronic liver disease and a substantial number of these patients have to undergo liver transplant. Unfortunately, the virus survives in other cells and eventually infects the new liver upon transplant. HCV infected patients have a higher mortality rate than non-HCV infected liver transplant patients at five years, likely due, at least in part, to accelerated HCV infection of the transplanted liver, leading to the recurrence of liver failure.

Immunosuppressive agents, or immunosuppressants, are invariably required for all allografts to blunt the recipient's immune response and minimize rejection. Use of immunosuppressants, however, has been linked to the increase in HCV virulence and in patient morbidity and mortality. This effect is especially pronounced in liver transplantation and is observed to a lesser extent in kidney transplantation.

Contradicting observations, however, have been widely reported with regard to some of the immunosuppressants, especially cyclosporine A (CsA). In some instances, CsA treatments are known to lead to an increase in virulence of HCA in the liver (see e.g. Everson, Impact of immunosuppressive therapy on recurrence of hepatitis C. Liver Transpl, 2002. 8(10 Suppl 1): p. S19-27), yet in other instances, CsA has been shown to inhibit HCV replication in vitro and has been used as a treatment for HCV infection. For example, Nakagawa et al. (Specific inhibition of hepatitis C virus replication by cyclosporin A. Biochem Biophys Res Commun 313(1):42-7. 2004) and Watashi et al. (Cyclosporin A suppresses replication of hepatitis C virus genome in cultured hepatocytes. Hepatology 38(5):1282-8. 2003) reported that CsA can inhibit HCV replication in vitro through a mechanism apparently unrelated to its immunosuppressive properties. Though CsA does not appear to control HCV effectively in liver transplant recipients, presumably due to its immunosuppressive effects, a study in Japan found that a six-month course of HCV treatment with a combination of CsA and alpha interferon was more effective at achieving sustained virological responses than interferon alone (42/76 [55%] vs. 14/44 [32%]; p=0.01) (Inoue et al., Combined interferon alpha2b and cyclosporin A in the treatment of chronic hepatitis C: controlled trial. J. Gastroenterol 38:567-72. 2003). Further research is focused on NIM811, a CsA analogue without immunosuppressive activity.

The inconsistency among the various reported research likely involves difference in study design, varying complexity of the patient population, such as differences in how patients respond to immunosuppressants, and other factors. The most likely cause of the inconsistency, however, is the high genetic heterogeneity of the HCV virus. Based upon phylogenetic analysis of the core, EI, and NS5 regions of the viral genome, the HCV virus has been classified into at least six genotypes and more than 30 subtypes dispersed throughout the world (Major and Feinstone, 1997, Hepatology 25: 1527-1538; Clarke, 1997, J. Genl. Virol 78: 2397-2410). It is believed that various genotypes or subtypes of HCV may be susceptible to inhibition by immunosuppressants such as cyclosporine A (CsA), while others may not. However, direct or specific correlation between the genotype of a HCV strain and its susceptibility to immunosuppressant treatment is lacking. As a consequence, currently, modifying CsA treatment of HCV in transplant patients is reactionary, with viral load or increased virulence as indicated by tissue destruction being the only indicators of failure of CsA treatment of HCV.

There is, therefore, a need to determine the susceptibility of a viral strain to an immunosuppressant in a patient, and to optimize immunosuppressant treatment regimens to prevent graft rejection without leaving the patient vulnerable to excessive morbidity and mortality from HCV infection. There is further a need for tools which physicians can use before and during CsA treatment to monitor development of CsA resistance by the virus, to predict and verify treatment efficacy, and to customize treatment.

### SUMMARY OF THE INVENTION

The present invention provides methods and compositions that employ determining changes or mutations or polymorphism in genomic sequences of HCV to determine the effectiveness of immunosuppressants, especially CsA, in treating HCV infection in general, and in liver transplant patients in particular. The invention further provides a kit to determine or monitor CsA resistance in HCV infected patient. Additionally, the present invention provides a method for treating HCV infection comprising a CsA treatment regimen, followed by testing the viral genome for CsA resistance and interferon susceptibility, and when appropriate an interferon treatment regimen.

According to one embodiment, the present invention provides a method for determining susceptibility of a HCV virus in a sample to CsA, the method comprising determining the nucleic acid sequence of the viral genome, and comparing it to that of a wild-type, CsA susceptible strain, wherein the existence of at least one mutation in the viral genome is indicative that the virus is resistant to CsA. Preferably, the mutations are in the regions of the viral genome that code for the viral proteins NS5A, or NS5B. Preferably, at least two mutations, one in NS5A and one in NS5B, are determined, and are more strongly indicative of increased CsA resistance. In a particularly preferred embodiment, detection of the specific amplicon sequences shown in Table 1 are indicative of CsA resistance. In particular, one or more mutations at positions, I259, A260, M269, S278, V284, L285, F288, L307, K309, K312, I317, D324, Y325, V343, V351, P355, R360, R361, K362, D393, C417, S419, E443, E446, V448, and C451 of NS5A; and A97, G104, L111, P133, V205, K254, Y276, Q309, T344, A348, S377, I412, V405, A435, E440, P460, Y524, L534, L536, P538, I539, G554, S556, L564, and S580 of NS5B are strongly indicative of increased CsA resistance.

In another embodiment, the present invention further provides a kit for detecting mutations in HCA genome, the kit comprising in a container a nucleic acid molecule, as described above, designed for detecting the mutation, and optionally at least one other component for carrying out such detection. Preferably, a kit comprises at least two oligonucleotides packaged in the same or separate containers. The kit may also contain other components such as instructional materials and reagents (e.g. hybridization buffer where the oligonucleotides are to be used as a probe and/or enzymes for PCR or RFLP) packaged in a separate container. Alternatively, where the oligonucleotides are to be used to amplify a target region, the kit may contain, preferably packaged in separate containers, a polymerase and a reaction buffer optimized for primer extension mediated by the polymerase, such as PCR.

The present invention further provides an array or a chip comprising a plurality of oligonucleotides that correspond to one or more mutations identified in Table 1.

The present invention further provides novel, CsA resistant viral replicons, such as those designated as “resistant” in Table 1, i.e. Amplicons 4, 5, 4-5A, 4-5B, 10 and 12. These CsA resistant viral replicons can be used for in vitro screening of anti-viral pharmaceutical compounds.

Thus in another embodiment, the present invention further provides screening methods for anti-viral compounds using the CsA resistant viral replicons or amplicons of the present invention. Specifically, the screening method comprises applying a candidate compound to a culture of cells that have been infected or transfected with a CsA resistant replicon of the present invention, and determining whether the candidate compound inhibits viral replication or viral protein expression, wherein a candidate that shows inhibitory effects is an antiviral compound. Candidate compounds may preferably be an RNA polymerase inhibitor, such as a nonnucleoside.

The present inventors further discovered that certain CsA-resistant HCV mutant strains may have increased susceptibility to interferon. Accordingly, the present invention provides a method for treating a HCV patient with CsA, followed by testing for the mutations, and when the interferon susceptible mutations are detected, treating the patient with interferon. While under CsA treatment HCV may evolve resistance to CsA, this resistance is coextensive of increased interferon susceptibility and a follow up treatment will help clear HCV from the patient.

### BRIEF DESCRIPTION OF FIGURES

FIG. 1 shows that CsA has the most anti-HCV activity of commonly used immunosuppressants.

FIG. 2 shows that inhibition of HCV replicon by both CsA and interferon increases with time.

FIG. 3 shows that mutations in NS5A/5B confer CsA resistance, and that exposure of the virus to CsA selects for CsA resistance. (A) Summary of mutations compared to pNNeo/3-5B (SI) replicon after CsA exposure. The 2nd and 3rd column indicate mutations found in NS5A and NS5B from 3 independent clones respectively. The wild-type amino acid and residue number are indicated on the top while the mutated residues found in CsA selected replicons are indicated below. The CsA IC90 value for each replicon is shown in the 4th column. (B) Frequency of mutations in NS5A and NS5B obtained during sequencing of 16 independent PCR fragments. (C) Comparison of the pNtat2ANeo/SI replicon with mutants Replicon 4, Replicon 4 5A and Replicon 4 5B. (D) Comparison of the CsA susceptibility of pNtat2ANeo/SI replicon with mutants Replicon 4 (also designated CsA-1s), Replicon 10 (also designated CsA-2s), and Replicon 12 (also designated CsA-3s). EN5.3 cells were stably transfected with the pNtat2ANeo/SI, Replicon 4, CsA-2s and CsA-3s replicons using G418 selection. Equal numbers of each replicon cell were seeded and treated with noncytotoxic concentrations of CsA for 7 days. On day 6, media, the cells were washed extensively and replaced with new media and CsA. Media aliquots were collected on day 7 and assayed for SEAP activity. Data are representative of three separate experiments.

FIG. 4 is the wild-type nucleic acid sequence of the NS5A-NS5B regions of the HCV viral genome.

FIG. 5 shows that Cyclophilin B interacts with both genotype 1a and mutant CsA-1s NS5B polymerases.

FIG. 6 shows that genotype 1b NS5A up-regulates viral translation, mutant 1b NS5A and genotype 1a NS5A do not.

### DETAILED DESCRIPTION OF THE INVENTION

The present inventors have isolated specific CsA-resistant mutants. These mutants reveal a proline rich region of the thumb of the polymerase that may require cyclophilin B for proper folding in certain strains of HCV. This region includes the coding sequence for HCV RNA dependent RNA polymerase (RdRp), the NS5B protein, which is a 68 kDa protein that catalyzes RNA synthesis during replication, and displays a classical Finger/Palm/Thumb motif observed in nucleic acid polymerases and a number of molecular interaction sites that can be targeted by specific inhibitors. This region is a known target for nonnucleoside inhibitors, see for example Howe et al, Novel nonnucleoside inhibitor of hepatitis C virus RNA-dependent RNA polymerase, Antimicrobial Agents & Chemotherapy. 48(12):4813-21 (2004).

Mutations in the Interferon Sensitivity Determining Region of NS5A have also been found to confer increased CsA resistance to HCV. These mutations suggest that CsA activity may be strain-dependant and explain clinical studies that both fail to show antiviral activity of CsA as well as studies that support the antiviral activity of CsA. Forward genetics were used to select HCV replicons that are less inhibited by CsA. These “CsA resistant sequences” were then used to determine the relationship between sequence variation of HCV and CsA sensitivity in vitro.

The present inventors have shown that the antiviral benefit of CsA varies according to variations of the HCV genome, and certain HCV strains display more cyclosporine sensitivity than others. Thus, the present invention provides method and compositions that employ determining changes or mutations or polymorphism in genomic sequences of HCV to determine the effectiveness of immunosuppressants, especially CsA, in treating HCV infection in general, and in liver transplant patients in particular. The invention further provides a kit to determine or monitor CsA resistance in HCV infected patient.

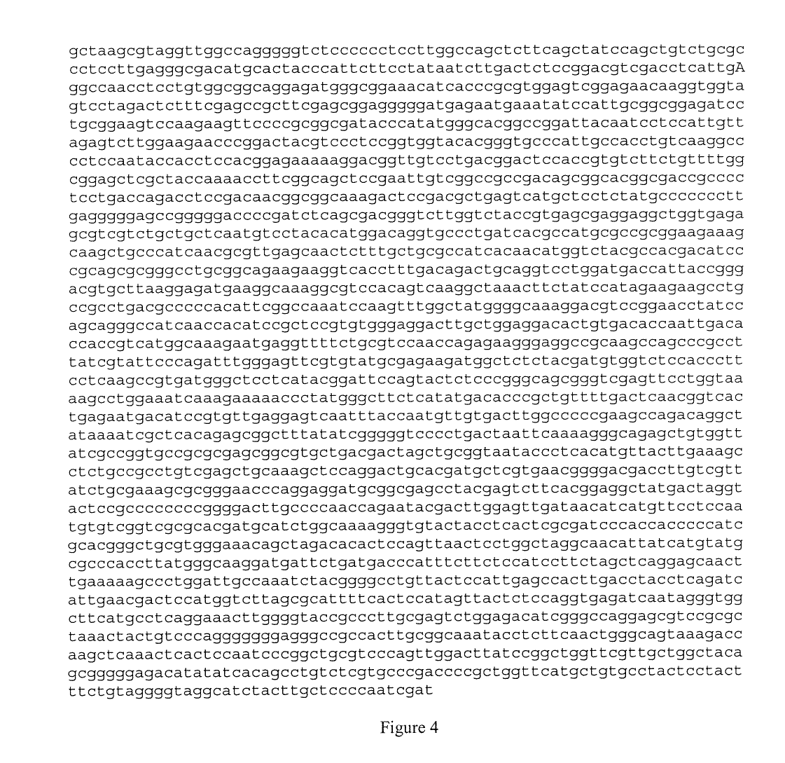

According to one embodiment, the present invention provides a method for determining susceptibility of a HCV virus in a sample to CsA, the method comprising determining the nucleic acid sequence of the viral genome, and comparing it to that of a wild-type, CsA susceptible strain, wherein the existence of at least one mutation in the viral genome is indicative that the virus is resistant to CsA. The present inventors have gained insight that the viral proteins NS5A, NS5B, or both, appear to be involved in conferring CsA resistance. Accordingly, in a preferred embodiment, the sequence of the viral genome that encodes these two proteins should be determined and compared to their respective wild-type sequences. In a more preferred embodiment, a region of about 700 amino acids (aa) corresponding to that shown in FIG. 4 is determined and compared to that of the wild type HCV, wherein the existence of at least one, preferably at least a plurality, of mutations, especially those shown in Table 1, is indicative that the virus is resistant to CsA.

In particular, one or more mutations at positions, I259, A260, M269, S278, V284, L285, F288, L307, K309, K312, I317, D324, Y325, V343, V351, P355, R360, R361, K362, D393, C417, S419, E443, E446, V448, and C451 of NS5A; and A97, G104, L111, P133, V205, K254, Y276, Q309, T344, A348, S377, I412, V405, A435, E440, P460, Y524, L534, L536, P538, I539, G554, S556, L564, and S580 of NS5B are strongly indicative of increased CsA resistance.

Preferably, at least two mutations, one in NS5A and one in NS5B, are determined, and are more strongly indicative of increased CsA resistance. In a particularly preferred embodiment, diction of the specific amplicon sequences shown in Table 1 are indicative of CsA resistance.

Many methods of collecting suitable samples from a patient for isolating the virus and determining the sequence of the viral genome are known in the art, for example, via RT-PCR and sequencing. Accordingly, the present invention also provides suitable PCR primers

The present invention also encompasses the complementary sequence corresponding to the polymorphism.

An objective of genotyping according to the present invention is to determine the nucleotide sequences at specific sites of interest in a specific sample of containing viral RNA. Many detection techniques are available and well-known to those skilled in the art.

For example, an oligonucleotide probe can be used for such a purpose. Preferably, the oligonucleotide probe will have a detectable label. Experimental conditions can be chosen such that if the sample contains a particular nucleotide at the position of interest, then the hybridization signal can be detected because the probe hybridizes to the corresponding nucleic acid strand in the sample, while if the sample DNA contains a mismatch, no hybridization signal is detected. Preferably, the viral RNA is reverse transcribed and the resultant DNA is used for detection purposes.

Similarly, PCR primers and conditions can be devised, whereby the oligonucleotide is used as one of the PCR primers, for analyzing nucleic acids for the presence of a specific sequence. Preferably, these are RT-PCR amplification of the viral RNA. Amplification may be used to determine the nucleotide identity at a specific position, by using a primer that is specific for a mutated or wild-type sequence. Alternatively, various methods are known in the art that utilize oligonucleotide ligation as a means of detecting polymorphisms, for examples see Riley et al (1990) Nucleic Acids Res. 18:2887-2890; and Delahunty et al (1996) Am. J. Hum. Genet. 58:1239-1246.

The detection method of the present invention may also be based on direct sequencing, or hybridization, or a combination thereof.

As used in the context of the present invention, the term “primer” refers to a single-stranded oligonucleotide capable of acting as a point of initiation of template-directed DNA synthesis under appropriate conditions (i.e., in the presence of four different nucleoside triphosphates and an agent for polymerization, such as, DNA or RNA polymerase or reverse transcriptase) in an appropriate buffer and at a suitable temperature. The appropriate length of a primer depends on the intended use of the primer but typically ranges from 15 to 30 nucleotides. Short primer molecules generally require cooler temperatures to form sufficiently stable hybrid complexes with the template. A primer need not reflect the exact sequence of the template but must be sufficiently complementary to hybridize with a template. The primer site, or priming site, is the area of the target DNA to which a primer hybridizes. The term primer pair means a set of primers including a 5′ upstream primer that hybridizes with the 5′ end of the DNA sequence to be amplified and a 3′, downstream primer that hybridizes with the complement of the 3′ end of the sequence to be amplified.

The term “probe” or “hybridization probe” denotes a defined nucleic acid segment (or nucleotide analog segment) which can be used to identify by hybridization a specific polynucleotide sequence present in samples, said nucleic acid segment comprising a nucleotide sequence complementary of the specific polynucleotide sequence to be identified. “Probes” or “hybridization probes” are nucleic acids capable of binding in a base-specific manner to a complementary strand of nucleic acid.

Hybridization may be performed in solution, or such hybridization may be performed when either the oligonucleotide probe or the target polynucleotide is covalently or noncovalently affixed to a solid support. Attachment may be mediated, for example, by antibody-antigen interactions, poly-L-Lys, streptavidin or avidin-biotin, salt bridges, hydrophobic interactions, chemical linkages, UV cross-linking baking, etc. Oligonucleotides may be synthesized directly on the solid support or attached to the solid support subsequent to synthesis. Solid-supports suitable for use in detection methods of the invention include substrates made of silicon, glass, plastic, paper and the like, which may be formed, for example, into wells (as in 96-well plates), slides, sheets, membranes, fibers, chips, dishes, and beads. The solid support may be treated, coated or derivatized to facilitate the immobilization of the allele-specific oligonucleotide or target nucleic acid. For screening purposes, hybridization probes of the polymorphic sequences may be used where both forms are present, either in separate reactions, spatially separated on a solid phase matrix, or labeled such that they can be distinguished from each other.

Hybridization may also be performed with nucleic acid arrays and subarrays such as described in WO 95/11995. The arrays would contain a battery of specific oligonucleotides representing each of the mutation sites of interest. Preferably, such an array will include at least 2 different oligonucleotide sequences, preferably at least one in the NS5A region, and one in the NS5B region.

The present invention further provides an array or a chip comprising a plurality of oligonucleotides that correspond to one or more mutations identified in Table 1.

Nucleic acid microarray technology, also known by other names including: DNA chip technology, gene chip technology, and solid-phase nucleic acid array technology, is well known to those of ordinary skill in the art. In general, an array of nucleic acid probes are fixed to a suitable substrate, to which target molecules, which are labeled with reporter molecules (e.g., radioactive, chemiluminescent, or fluorescent tags such as fluorescein, Cye3-dUTP, or Cye5-dUTP), are hybridized, and target-probe hybridization is evaluated. A probe with a nucleic acid sequence that matches the target sequence will, in general, result in detection of a reporter-molecule signal. Many components and techniques utilized in nucleic acid microarray technology are presented in The Chipping Forecast, Nature Genetics, Vol. 21, January 1999, the entire contents of which is incorporated by reference herein.

Nucleic acid microarray substrates suitable for the present invention may include but are not limited to glass, silica, aluminosilicates, borosilicates, metal oxides such as alumina and nickel oxide, various clays, nitrocellulose, or nylon. A glass substrate is generally preferred.

In one embodiment, the microarray substrate may be coated with a compound to enhance synthesis of the probe on the substrate. In another embodiment, coupling agents or groups on the substrate can be used to covalently link the first nucleotide or olignucleotide to the substrate.

The oligonucleotide sequence on the array will usually be at least about 12 nt in length, or may extend into the flanking regions to generate fragments of 100 to 200 nt in length. For examples of arrays, see Ramsay (1998) Nat. Biotech. 16:4044; Hacia et al. (1996) Nature Genetics 14:441-447; Lockhart et al. (1996) Nature Biotechnol. 14:1675-1680; and De Risi et al. (1996) Nature Genetics 14:457-460. As well-known to those ordinarily skilled in the art, the presence or absence of hybridization signals, optionally in combination of the signal strength, will determine the presence or absence of which of the mutations, and whether the virus in the sample has increased resistance to CsA.

The existence of CsA resistance mutations in the viral genome of the sample may also be determined using a mismatch detection technique, including but not limited to the RNase protection method using riboprobes (Winter et al., Proc. Natl. Acad. Sci. USA 82:7575, 1985; Meyers et al., Science 230:1242, 1985) and proteins which recognize nucleotide mismatches, such as the E. coli mutS protein (Modrich, P. Ann. Rev. Genet. 25:229-253, 1991). Alternatively, mutations can be identified by single strand conformation polymorphism (SSCP) analysis (Orita et al., Genomics 5:874-879, 1989; Humphries et al., in Molecular Diagnosis of Genetic Diseases, R. Elles, ed., pp. 321-340, 1996) or denaturing gradient gel electrophoresis (DGGE) (Wartell et al., Nucl. Acids Res. 18:2699-2706, 1990; Sheffield et al., Proc. Natl. Acad. Sci. USA 86:232-236, 1989).

A polymerase-mediated primer extension method may also be used to identify the mutaion(s). Several such methods have been described in the patent and scientific literature and include the “Genetic Bit Analysis” method (WO 92/15712) and the ligase/polymerase mediated genetic bit analysis (U.S. Pat. No. 5,679,524). Related methods are disclosed in WO 91/02087, WO 90/09455, WO 95/17676, U.S. Pat. Nos. 5,302,509, and 5,945,283. Extended primers containing a mutation may be detected by mass spectrometry as described in U.S. Pat. No. 5,605,798. Another primer extension method is allele-specific PCR (Ruao et al., Nucl. Acids Res. 17:8392, 1989; Ruao et al., Nucl. Acids Res. 19, 6877-6882, 1991; WO 93/22456; Turki et al., J. Clin. Invest. 95:1635-1641, 1995).

When nucleic acid amplification method are used, a detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

It is readily recognized by those ordinarily skilled in the art that in order to maximize the signal to noise ratio, in probe hybridization detection procedure, the site where the mutation is should at the center of the probe fragment used, whereby a mismatch has a maximum effect on destabilizing the hybrid molecule; and in a PCR detection procedure, the polymorphic site should be placed at the very 3′-end of the primer, whereby a mismatch has the maximum effect on preventing a chain elongation reaction by the DNA polymerase. The location of nucleotides in a polynucleotide with respect to the center of the polynucleotide is described herein in the following manner. When a polynucleotide has an odd number of nucleotides, the nucleotide at an equal distance from the 3′ and 5′ ends of the polynucleotide is considered to be “at the center” of the polynucleotide, and any nucleotide immediately adjacent to the nucleotide at the center, or the nucleotide at the center itself is considered to be “within 1 nucleotide of the center.” With an odd number of nucleotides in a polynucleotide any of the five nucleotides positions in the middle of the polynucleotide would be considered to be within 2 nucleotides of the center, and so on. When a polynucleotide has an even number of nucleotides, there would be a bond and not a nucleotide at the center of the polynucleotide. Thus, either of the two central nucleotides would be considered to be “within 1 nucleotide of the center” and any of the four nucleotides in the middle of the polynucleotide would be considered to be “within 2 nucleotides of the center,” and so on.

Alternatively, the relevant portion of the relevant genetic locus of the sample of interest may be amplified via RT-PCR and directly sequenced. PCR and sequencing techniques are well known in the art and reagents and equipments are readily available commercially. The identified of the polymorphic site in the amplified fragment may also identified by RFLP, according to method and techniques well-known to those skilled in the art.

Alternatively, an invasive signal amplification assay, as described in e.g. U.S. Pat. No. 5,422,253 and Lyamichev et al., 2000, Biochemistry 39:9523-9532, both incorporated herein by reference in their entirety, may be used for detecting the mutations of interest. This assay takes advantage of enzymes such as the 5′ nuclease activity of a DNA polymerase or the gene 6 product from bacteriophage T7 in their ability to cleave polynucleotide molecules by recognizing specific structures instead of specific sequences. A single stranded target molecule is annealed to a pilot oligonucleotide such that the 5′ end of the pilot forms a duplex with the target molecule. If the 3′ end of the pilot oligonucleotide does not pair with the target, a 3′ arm is formed. When exposed to a cleavage agent such as a DNA polymerase having a 5′ nuclease activity or the gene 6 product from bacteriophage T7, the target molecule is cleaved in the 5′ region, one nucleotide into the duplex adjacent to the unpaired region of the target. If a cut in a double-stranded molecule is required, the double-stranded molecule is denatured. Because this unpaired 3′ arm can be as short as one nucleotide, this assay can be used for detecting a single-nucleotide difference, e.g. in the context of SNP detection. The pilot oligonucleotide is designed such that it pairs perfectly with one allele, but has a 3′, single nucleotide mismatch with another allele. Cleavage only occurs if there is a mismatch between the target molecule and the pilot. To achieve signal amplification, the above invasive reaction is modified such that cleavage occurs on the pilot oligonucleotide. Two oligonucleotides are annealed in an adjacent manner to the target molecule. The resulting adjacent duplexes overlaps by at least one nucleotide to create an efficient substrate, called the overlapping substrate, for the 5′ nucleases. The 5′ end of the downstream oligonucleotide, also called the probe, contains an unpaired region termed the 5′ arm (Lyamichev et al., 1993, Science 260:778-783.) or flap (Harrington and Lieber, 1994, EMBO J. 13: 1235-1246) that is not required for the enzyme activity; however, very long arms can inhibit cleavage (Lyamichev et al., 1993, Science 260:778-783). Specific cleavage of the probe, termed invasive cleavage (Lyamichev et al., 1999, Nat. Biotechnol. 17: 292-296; Kwiatkowski et al., 1999, Mol. Diagn. 4, 353-364.), occurs at the position defined by the 3′ end of the upstream oligonucleotide, which displaces or “invades” the probe. If the overlap between the adjacent oligonucleotides is only one nucleotide, cleavage takes place between the first two base pairs of the probe, thus releasing its 5′ arm and one nucleotide of the base paired region (Lyamichev et al., 1999, Proc. Natl. Acad. Sci. U.S.A. 96: 6143-6148, and Kaiser et al., 1999, J. Biol. Chem. 274:21387-21394). If the upstream oligonucleotide and the probe are present in large molar excess over the target nucleic acid, and invasive cleavage is carried out near the melting temperature of the probe, a cut probe can rapidly dissociate, and an intact probe will anneal to the target more frequently than will a cut probe, thus initiating a new cycle of cleavage. This allows multiple probes to be cut for each target molecule under isothermal conditions, resulting in linear signal amplification with respect to target concentration and time (Lyamichev et al., 1999, Nat. Biotechnol. 17: 292-296).

The present invention further provides a kit for detecting mutations in HCA genome in a sample, the kit comprising in a container a nucleic acid molecule, as described above, designed for detecting the mutation, and optionally at least one other component for carrying out such detection. Preferably, a kit comprises at least two oligonucleotides packaged in the same or separate containers. The kit may also contain other components such as instructional materials and reagents (e.g. hybridization buffer where the oligonucleotides are to be used as a probe and/or enzymes for PCR or RFLP) packaged in a separate container. Alternatively, where the oligonucleotides are to be used to amplify a target region, the kit may contain, preferably packaged in separate containers, a polymerase and a reaction buffer optimized for primer extension mediated by the polymerase, such as PCR.

Further, the present invention provides a method of monitoring the development of CsA resistance in a HCV patient, especially in liver transplant patients afflicted by HCV infection. HCV is a highly mutatable virus, and it is known that under selection pressure of CsA treatment, resistant or tolerant mutations occur and accumulate in patients. Using the method of the present invention, a physician will be enabled to manage antiviral and immunosuppressant treatment of the patient according to the effectiveness of CsA on HCV, for example in determining whether CsA should be used at all, alone, or in combination with other antiviral drugs and/or immunosuppressants.

The present invention further provides novel, CsA resistant viral replicons, such as those designated as “resistant” in Table 1, including Amplicons 4, 5, 4-5A, 4-% b, 10 and 12. These CsA resistant viral replicons can be used for in vitro screening of anti-viral pharmaceutical compounds.

Thus the present invention further provides screening methods for anti-viral compounds using the CsA resistant viral replicons or amplicons of the present invention. Specifically, the screening method comprises applying a candidate compound to a culture of cells that have been infected with a CsA resistant replicon of the present invention, and determining whether the candidate compound inhibits viral replication or viral protein expression, wherein a candidate that shows inhibitory effects is an antiviral compound. Candidate compounds may preferably be a RNA polymerase inhibitor, such as a nonnucleoside.

Many methods for detecting viral replication and viral protein expression are known to those skilled in the art. For example, the method may be a plaque assay, based on the formation of plaques due to host cell death, or cells in a dying process or detachment. Viral gene expression may be detected following a suitable staining procedure with a suitable antibody, for example (Duca K A, et al, Quantifying viral propagation in vitro: toward a method for characterization of complex phenotypes. Biotechnol Prog. 2001 November-December; 17(6):1156-65.) Observable indication of viral replication or viral gene expression may also be a host cell reaction to the virus infection. Virus infections can activate cellular defensive responses such as interferon pathways. Reporters that are linked to infection-mediated activation of such responses may indicate host cell reactions to virus infection. The observable indication may further be the expression of a transgene engineered into a HCV viral construct, such as a gene encoding a fluorescent protein whose expression can be easily monitored or quantified. For example, the host cell may be modified to carry a reporter enzyme whose expression is driven by a viral or an inducible promoter (see e.g. Wang, et al, A cell line that secretes inducibly a reporter protein for monitoring herpes simplex virus infection and drug susceptibility. Journal of Med. Virol. 2002 December; 68(4):599-605).

In another aspect, the present inventors discovered that the CsA resistant replicons contain mutations in the region of NS5A sequence from amino acid 250 to amino acid 315, which contains the known protein kinase R(PKR) binding site, 237-306. Specifically, Replicon 4, one of the most CsA resistant replicons, contains 6 total mutations in NS5A, four of which is in this region. In particular, it contains a mutation at position F288. It has been known that mutations at this region correlate to increased interferon susceptibility, see for example, Pascu et al., Sustained virological response in hepatitis C virus type 1b infected patients is predicted by the number of mutations within the NS5A-ISDR: a meta-analysis focused on geographical differences. Gut, 2004. 53(9): p. 1345-51. This suggests that the mutants arising under a CsA selection pressure may have increased susceptibility to interferon.

Accordingly, the present invention further provides a method for treating a HCV patient with CsA, followed by testing for the mutations, and treating the patient with interferon. While under CsA treatment HCV may evolve resistance to CsA, this resistance is coextensive of increased interferon susceptibility and a follow up treatment will help clear HCV from the patient.

### EXAMPLES

### Example 1

### Measuring HCV Replicon Amount in the Presence of Immunosuppressants

An established replicon model is available to study HCV resistance to interferon and immunosuppressants. In this model, a dicistronic RNA is transfected into cells which encodes the viral replication proteins with or without the structural genes while the second cistron encodes a selectable marker such as neomycin (Blight et al., Efficient initiation of HCV RNA replication in cell culture, Science, 2000. 290:1972-4; Ikeda et al., Selectable subgenomic and genome-length dicistronic RNAs derived from an infectious molecular clone of the HCV-N strain of hepatitis C virus replicate efficiently in cultured Huh7 cells. J. Virol, 2002. 76:2997-3006). This system allows autonomous replication of the viral RNA in a sequence specific manner that predicts efficacy of both current and novel therapy, even though infectious viral particles are not produced.

Different genotype 1b isolates or replicons have been reported to be CsA sensitive (Nakagawa et al., Suppression of hepatitis C virus replication by cyclosporin a is mediated by blockade of cyclophilins, Gastroenterology, 2005, 129:1031-41, Watashi et al., Cyclophilin B is a functional regulator of hepatitis C virus RNA polymerase, Mol Cell, 2005, 19: 111-22).

Two different assays were used to measure the amount of viral replicon RNA in cells exposed to immunosuppressants. The first is a direct real time RT-PCR quantification of the replicon RNA using primers and probes complimentary to HCV as well as a cellular target. Briefly, cells are lysed for RNA isolated using Trizol (Invitrogen) and real time RT-PCR performed (Stangl et al., Effect of antimetabolite immunosuppressants on Hepatitis C Virus. Transplantation, 2004, 77:562-567). The second assay takes advantage of a reporter system where the viral replicon RNA encodes both the neomycin resistance gene, and the HIV tat protein. The replicon was stably transfected into a modified cell line, (EN5-3) engineered to secrete alkaline phosphatase (SEAP) into the media when tat is expressed by the viral replicon. Chemiluminescence assays of SEAP in the media correlate linearly with viral replicon levels (Yi et al. Subgenomic hepatitis C virus replicons inducing expression of a secreted enzymatic reporter protein. Virology, 2002. 304(2): p. 197-210). This system allows quantification of viral RNA intracellular replicon levels by sampling the media over time without needing to lysis the cells.

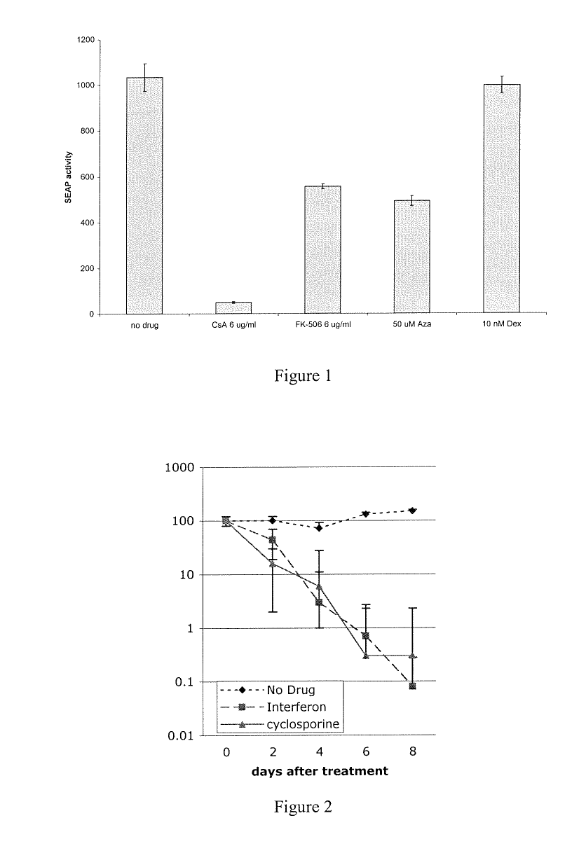

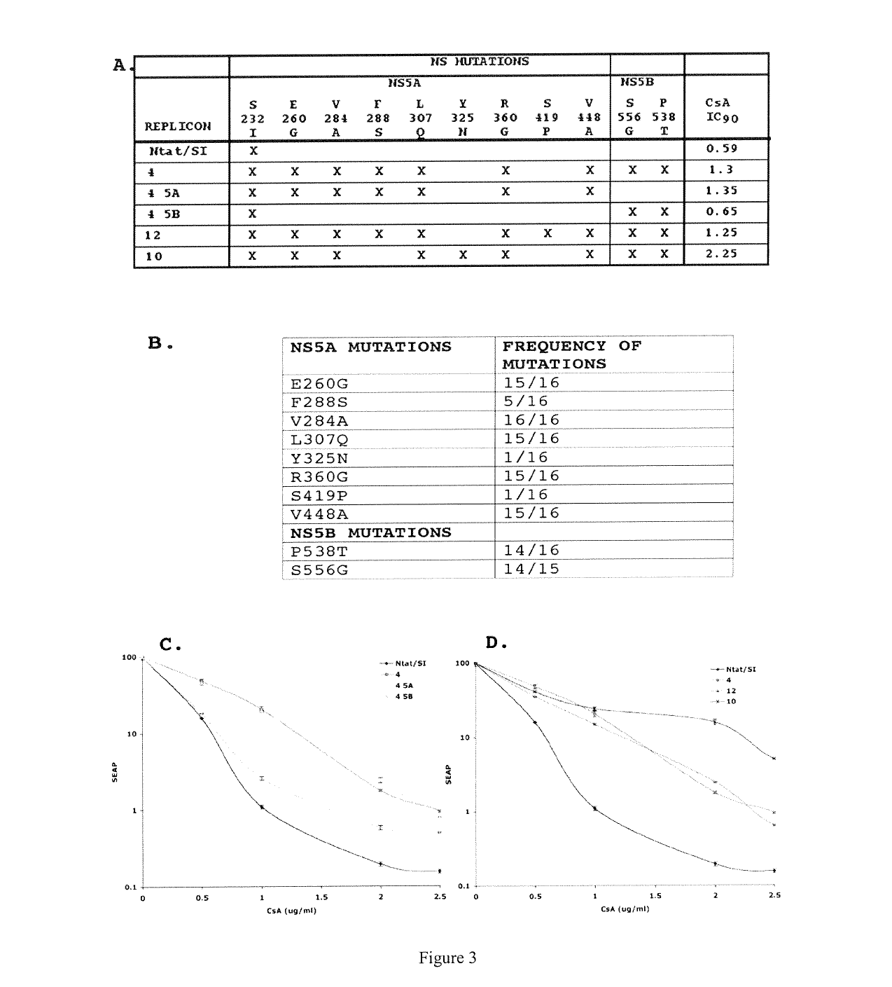

The effect of all commonly used immunosuppressants on the HCV replicon: AZA, mycophenolate (Stangl et al., 2004, supra) corticosteroids, CsA, and Tac has been systematically evaluated. Similar studies with related Flaviviridae including Bovine Viral Diarhea Virus and Yellow Fever Virus (FIG. 1 and data not shown) have also been performed. Genotype 1bN of HCV is much more sensitive to CsA (dose response shown in FIG. 3) than other Flaviviridae.

### Example 2

### CsA has the Most Anti-HCV Activity of Commonly Used Immunosuppressants

EN5-3 cells harboring a modified HCV replicon which induces secretion of SEAP from these cells were cultured in the presence of no drug, CsA, FK506, Aza, or dexamethasone for 4 days. Maximally achievable concentrations without cell toxicity are shown. Media from each tissue culture dish was then assayed for the presence of SEAP by Phospha-Light Chemiluminescent Reporter Assay (Tropix). 50 μM AZA resulted in ˜10% fewer cells otherwise not toxicity was observed. FIG. 1 shows that CsA has the most anti-HCV activity of commonly used immunosuppressants.

In fact, as shown in FIG. 2, the antiviral effect of both CsA and interferon accumulates over time, with similar kinetics, which is not the case for AZA. Huh7 cells bearing the 1bN replicon (Ikeda et al., Selectable subgenomic and genome-length dicistronic RNAs derived from an infectious molecular clone of the HCV-N strain of hepatitis C virus replicate efficiently in cultured Huh7 cells. J. Virol, 2002. 76(6): p. 2997-3006) were incubated with no drug, 50 U/ml Interferon or 1 ug/ml CsA. After the indicated number of days cells were lysed and Real Time RT-PCR was performed to quantitate both viral RNA and cellular GADPH mRNA, using a method previously reported (Stangl et al., Effect of antimetabolite immunosuppressants on Hepatitis C Virus. Transplantation, 2004. 77(4): p. 562-567). These results confirmed that CsA has a potent antiviral activity that is proportional to exposure.

### Example 3

### Cyclosporine Resistant Replicons can be Selected in Cell Culture and Mutations in NS5A/5B Confer CsA Resistance

The HCV 1bN replicon was continuously cultivated in the presence of either 4 or 10 μg/ml of CsA, and 1.0 mg/ml geneticin for over 4-12 weeks. CsA resistant replicons were selected with both the neomycin reporter replicon (monitored by Real Time RT-PCR) and the neomycin/SEAP reporter replicon (monitored by SEAP accumulation in the media, see FIG. 1). Tat expressing reporter construct was serially passaged the in the presence of CsA, as monitoring SEAP accumulation does not require lysing the cells to obtain RNA. It is conceivable that mutations would accumulate that allow for tat expression/alkaline phosphatase secretion without actual viral replicon replication. Therefore the 1bN replicon was also selected without the tat/SEAP reporter system. In both cases, after only 3-4 weeks of cultivation in the presence of CsA, a 200-800% difference in the CsA susceptibility was observed.

Additional data suggest that cultivation of the replicon in the presence of 4 μg/ml CsA also produced resistant replicon, after a longer exposure (8 weeks).

Sequence analysis of replicons was conducted that have been selected with 10 μg/ml after 4 weeks. Sequencing of these pools revealed some wild-type virus as well as multiple synonymous mutations throughout the viral genomic RNA. In both independent selections though there was a clustering of nonsynonymous mutations in the carboxy terminal domains of NS5A and NS5B, two proteins that themselves interact (Shirota et al., Hepatitis C virus (HCV) NS5A binds RNA-dependent RNA polymerase (RdRP) NS5B and modulates RNA-dependent RNA polymerase activity. J. Biol. Chem., 2002, 277:11149-55).

A 1bN HCV replicon with a single NS5A cell culture adaptation mutation was selected in Huh7 cells in the presence of 10 μg/ml CsA and geneticin for 3 weeks. The population of replicons was then amplified, cloned and sequenced. Since the majority of nonsynonymous mutations were found in NS5A and NS5B, three fragments representing independent clones of that region were engineered back into a replicon bearing the preCsA exposure 1bN sequence (pNtat2ANeo/SI replicon which induces secreted alkaline phosphotase (SEAP) to levels proportional to replicon RNA (Yi and Lemon et. al., 2002, Subgenomic hepatitis C virus replicons inducing expression of a secreted enzymatic reporter protein. Virology 304:197-210).

CsA passage experiments—Huh7 cells carrying the pNNeo/3-5B(SI) replicon were passaged in the presence of 10 μg/ml of CsA (Sigma) and 1 mg/ml of G418 for a period of three weeks. Control cells containing the pNNeo replicon were passaged in the presence of 6418 without CsA. Cells were maintained below a confluency of 70-80%.

Amplification of replicon RNA by RT-PCR and cloning of amplified DNA fragments—Total RNA was isolated using TRIZOL and reverse transcribed using Superscript II/III (Invitrogen). cDNA was amplified by PCR using Taq (Invitrogen) for the entire replicon, and later with Pfu Turbo (Stratagene) for the Blp1-Cla1 fragments. PCR products were subcloned into TOPO 2.1 TA vectors and sequenced. Sequencing data was obtained from independent cDNA amplications and sequencing of 5-13 independent clones. TOPO 2.1 TA clones containing NS5A to the NS5B end were digested with BlpI and ClaI and swapped into the original construct pNtat2ANeo/SI. The constructs was designated CsA-1s (Replicon 4), 2 (Replicon 10) and 3 (Replicon 12). For construct CsA-1s 5A (Replicon 4 5A), construct CsA-1s was cut with Blp1 and BstXI and inserted into the original pNtat2ANeo/SI. In the case of construct CsA-1s 5B (Replicon 4 5B), the BstXI-ClaI portion of CsA-1s was inserted back into the pNtat2ANeo/SI construct.

A summary of mutations compared to pNNeo/3-5B (SI) replicon after CsA exposure is shown in FIG. 3A. The 2nd and 3rd column indicate mutations found in NS5A and NS5B from 3 independent clones respectively. The wild-type amino acid and residue number are indicated on the top while the mutated residues found in CsA selected replicons are indicated below. The CsA IC90 value for each replicon is shown in the 4th column. FIG. 3B shows the frequency of mutations in NS5A and NS5B obtained during sequencing of 16 independent PCR fragments. FIG. 3C shows a comparison of the CsA susceptibility of pNtat2ANeo/SI replicon with Replicons 4, 10 and 12.

EN5.3 cells were stably transfected with the pNtat2ANeo/SI, Replicons 4, 10 and 12 using G418 selection. Equal numbers of each replicon cell were seeded and treated with noncytotoxic concentrations of CsA for 7 days. On day 6, media, the cells were washed extensively and replaced with new media and CsA. Media aliquots were collected on day 7 and assayed for SEAP activity. Data are representative of three separate experiments. (D) Comparison of the pNtat2ANeo/SI replicon with mutants Replicons 4, 4 5A and 4 5B. Mutant NS5A and NS5B from the CsA-1s mutant were individually cloned into the pNtat2ANeo/SI replicon, to produce replicons 4 5A and 4 5B respectively. Comparison of the resistance of these replicons to CsA is described in FIG. 3 (C).

Table 1 shows the mutations observed during these selections in NS5A and NS5B. The left-most column shows the amino acid residue identity at a particular position, and the second column shows its corresponding codon. The mutations are denoted with its resultant codon after the mutation, plus the amino acid it encodes. For example, for Amplicon 2, at position 260, the original codon was GCG, encoding an alanine, as a result of the mutation, the codon became GGG, encoding a glycine (GGG-G). Every mutation shown in the tables was recovered multiple times using a low-fidelity enzyme, and at least once independently with a high fidelity polymerase.

All amplicons shown in Table 1 are selected (designated S) after exposure to CsA. Amplicons 4, 5, 4-5A, 4-5B, 10 and 12 have been tested and proven to have increased resistance over the wild type strain (designated R). Specifically, the replicon RNA synthesized by T7 polymerase, cloned back into the replicon cDNA, and transfected back into cells to determine if they confer CsA resistance. For example, mutants NS5A and NS5B from the CsA-1s mutant were individually cloned into the pNtat2ANeo/SI replicon. These replicons were called 4 5A and 4 5B respectively.

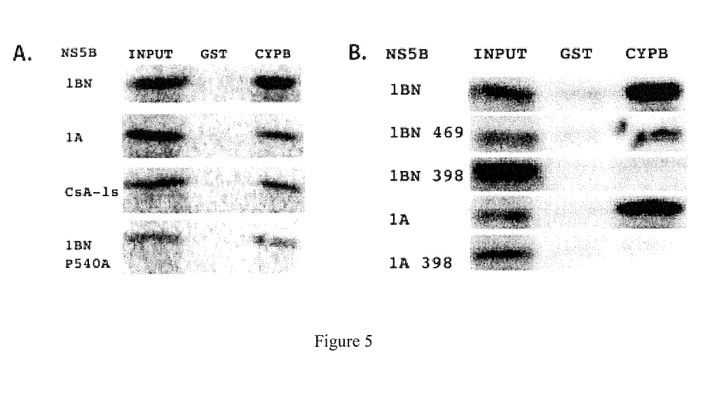

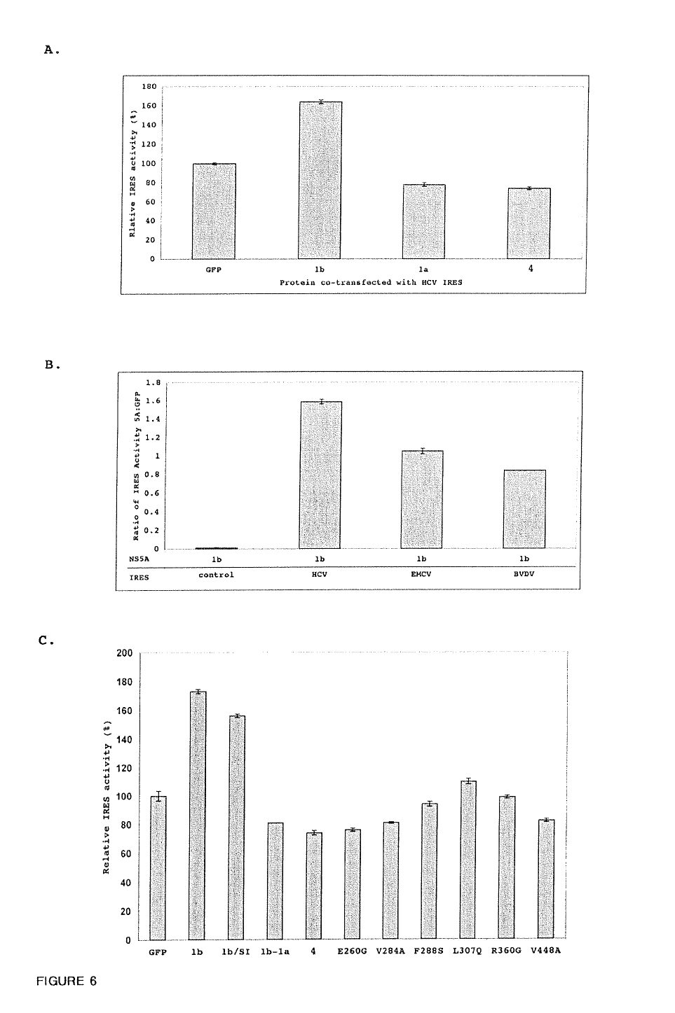

The wild-type sequence is shown in FIG. 5.

### Example 4

### Cyclophilin B Interacts with Both Genotype 1A and Mutant CsA-1s NS5B (Replicon 4 5B) Polymerases

A glutathione S-transferase (GST) pull down assay was performed as follows. The 1BN NS5B, 1A NS5B, CsA-1s NS5B and an NS5B with a proline to alanine change at position 540 were in vitro translated and S³⁵ labeled. The translated products were incubated with 0.5 mM GST alone or recombinant GST-CypB fusion proteins. After extensive washing, the samples were loaded on an SDS PAGE gel. Input represents 1/10th of the amount used in the GST/GST-CypB pull down assay. The results are shown in FIG. 5(a).

The CypB binding region of NS5B was mapped. Specifically, mutant truncates of the 1BN and 1A NS5B proteins were made by inserting stop codons at position at 521, 494, 469 and 398, but only the latter two are shown, since this is where the biggest change occurs. The truncated mutant NS5B's together where compared with the wild type 1BN and 1A NS5B's in the GST down assay as described above.

TABLE 1
Mutations in NS5A and NS5B

| WILD TYPE | | | | 4- | 4- | | | | | | | | | | | |
|---|---|---|---|---|---|---|---|---|---|---|---|---|---|---|---|---|
| CODON | | 4 | 5 | 5A | 5B | 12 | 10 | 2 | 3 | 9 | 11 | 13 | 6 | 14 | 7 | 8 |
| I259 | ATT | | | | | | | | | | | | | | ACT- | |
| | | | | | | | | | | | | | | | T | |
| A260 | GCG | GGG- | | GGG- | | GGG- | GGG- | GGG- | GGG- | GGG- | GGG- | | | | | |
| | | G | | G | | G | G | G | G | G | G | | | | | |
| M269 | ATG | | | | | | | | ACG- | | | | | | | |
| | | | | | | | | | T | | | | | | | |
| S278 | TCG | | CCG- | | | | | | | | | | | | | |
| | | | P | | | | | | | | | | | | | |
| V284 | GTC | GCC- | | GCC- | | GCC- | GCC- | GCC- | GCC- | GCC- | GCC- | | | | | |
| | | A | | A | | A | A | A | A | A | A | | | | | |
| L285 | CTA | | | | | | | | | | | | | CCA- | | |
| | | | | | | | | | | | | | | P | | |
| F288 | TTC | TCC- | | TCC- | | TCC- | | TCC- | | | | | | | | TCC- |
| | | S | | S | | S | | S | | | | | | | | S |
| L307 | CTG | CAG- | | CAG- | | CAG- | CAG- | | CAG- | CAG- | CAG- | | | | | |
| | | Q | | Q | | Q | Q | | Q | Q | Q | | | | | |
| K309 | AAG | | GAG- | | | | | | | | | | | | | |
| | | | E | | | | | | | | | | | | | |
| K312 | AAG | | | | | | | | | AAC- | | | | | | |
| | | | | | | | | | | N | | | | | | |
| I317 | ATA | | ATG- | | | | | TTA- | | | | | | | | |
| | | | M | | | | | L | | | | | | | | |
| D324 | GAT | | | | | | | | | | | | GAG- | | | |
| | | | | | | | | | | | | | E | | | |
| Y325 | TAC | | | | | | AAC- | | | | | | | | | |
| | | | | | | | N | | | | | | | | | |
| V343 | GTA | | GTG- | | | | | | | | | | GTG- | | | |
| | | | V | | | | | | | | | | V | | | |
| V351 | GTC | | | | | | | | | | GCC- | GCC- | | GCC- | GCC- | |
| | | | | | | | | | | | A | A | | A | A | |
| P355 | CCA | | | | | | | | | | | | | | TCA- | |
| | | | | | | | | | | | | | | | | S |
| R360 | CGG | GGG- | | GGG- | | GGG- | GGG- | | GGG- | GGG- | GGG- | | | | | |
| | | G | | G | | G | G | | G | G | G | | | | | |
| R361 | AGA | | AGG- | | | | | | | | | | AGG- | | | |
| | | | R | | | | | | | | | | R | | | |
| K362 | AAA | | | | | AAG- | | | | | | | | | | |
| | | | | | | K | | | | | | | | | | |
| D393 | GAC | | | | | | | GGC- | | | | | | | | |
| | | | | | | | | G | | | | | | | | |
| C417 | TGC | | | | | | | | | CGC- | | | | | | |
| | | | | | | | | | | R | | | | | | |
| S419 | TCT | | | | | CCT- | | | | | | | | | | |
| | | | | | | P | | | | | | | | | | |
| E443 | GAG | | GTG- | | | | | | | | | | GTG- | | | |
| | | | V | | | | | | | | | | V | | | |
| E446 | GAG | | | | | | | | | | | GGG- | | | | |
| | | | | | | | | | | | | G | | | | |
| V448 | GTC | GCC- | | GCC- | | GCC- | GCC- | | GCC- | GCC- | GCC- | | | | | |
| | | A | | A | | A | A | | A | A | A | | | | | |
| C451 | TGC | | TGT- | | | | | | | | | | TGT- | | | |
| | | | C | | | | | | | | | | C | | | |
| A97 | GCC | | | | | | | | | | GCT- | | | | | |
| | | | | | | | | | | | A | | | | | |
| G104 | GGG | | | | | | | | | | | | | | | AGG- |
| | | | | | | | | | | | | | | | | R |
| L111 | CTA | | | | | | | | | | | CTG- | | | | |
| | | | | | | | | | | | | L | | | | |
| P133 | CCA | | | | | | | | | | | | | | | CCC- |
| | | | | | | | | | | | | | | | | P |
| V205 | GTA | GTT- | | | GTT- | | GTT- | GTT- | GTT- | GTT- | GTT- | | | | | |
| | | V | | | V | | V | V | V | V | V | | | | | |
| K254 | AAA | | AGA- | | | | | | | | | | | | AGA- | |
| | | | R | | | | | | | | | | | | R | |
| Y276 | TAT | | | | | | | | | | | | | | TAC- | |
| | | | | | | | | | | | | | | | Y | |
| Q309 | CAG | | | | | | | | CAA- | | | | | | | |
| | | | | | | | | | Q | | | | | | | |
| T344 | ACT | | | | | | ACC- | | ACC- | | ACC- | | | | | |
| | | | | | | | T | | T | | T | | | | | |
| A348 | GCC | | | | | | | GCT- | | | | | | | | |
| | | | | | | | | A | | | | | | | | |
| S377 | TCT | | | | | | | | | | | | TTT- | | | |
| | | | | | | | | | | | | | F | | | |
| I412 | ATT | | | | | | | | | | CTT- | | | | | |
| | | | | | | | | | | | F | | | | | |
| V405 | GTT | GTC- | | | GTC- | | | | | | | | | | | |
| | | V | | | V | | | | | | | | | | | |
| A435 | GCT | GCG- | | | GCG- | GCG- | GCG- | | GCG- | GCG- | GCG- | | | GTT- | | |
| | | A | | | A | A | A | | A | A | A | | | V | | |
| E440 | GAA | | | | | | | | | | | | | GAG- | | |
| | | | | | | | | | | | | | | E | | |
| P460 | CCT | | | | | | CCC- | | | | | | | | | |
| | | | | | | | P | | | | | | | | | |
| Y524 | TAC | | | | | | | | | | | | | TGC- | | |
| | | | | | | | | | | | | | | C | | |
| L534 | CTC | | | | | | | TTC- | | | | | | | | |
| | | | | | | | | F | | | | | | | | |
| L536 | CTC | | | | | | | | | CTA- | | | | | | |
| | | | | | | | | | | L | | | | | | |
| P538 | CCA | ACA- | | | ACA- | ACA- | ACA- | | ACA- | ACA- | ACA- | | | | | |
| | | T | | | T | T | T | | T | T | T | | | | | |
| I539 | ATC | | | | | | | | | | | | GTC- | GTC- | | |
| | | | | | | | | | | | | | V | V | | |
| G554 | GGC | | | | | | | | | | | | GAC- | | | |
| | | | | | | | | | | | | | D | | | |
| S556 | AGC | GGC- | | | GGC- | GGC- | GGC- | | GGC- | GGC- | GGC- | | | | | |
| | | G | | | G | G | G | | G | G | G | | | | | |
| L564 | CTG | | | | | | | | | | | | ATG- | | | |
| | | | | | | | | | | | | | M | | | |
| S580 | TCT | | TCC- | | | | | | | | | | | | | |
| | | | S | | | | | | | | | | | | | |
| Ph. | SEN. | R | R | R | R | R | R | S | S | S | S | S | S | S | S | S |

### Example 5

### Genotype 1b NS5A Up-Regulates Viral Translation, Mutant 1b NS5A and Genotype 1a NS5A do not

GFP, 1BN-NS5A, 1A NS5A, and Replicon 4 NS5A were individually transfected into Huh7 cells, each in combination with a dual-luciferase HCV IRES reporter construct. The Firefly luciferase translation was driven by the HCV Internal Ribosome Entry Sequence (cap independent, IRES mechanism), while the Renilla luciferase was driven by the normal cap dependant mechanisms. Cell lysates were collected 48 h post-transfection and subjected to dual-luciferase assays. The relative IRES activities were expressed as the ratio of Firefly to Renilla luciferase activity. The activity of HCV IRES in GFP transfected cells was arbitrarily taken as 100% with the activities of the IRES under all NS5A species expressed relative to this. The results are shown in FIG. 6(A)

FIG. 6(B) shows that NS5A up-regulates HCV IRES but not other viral IRES containing constructs. GFP and 1BN-NS5A were separately transfected into Huh7 cells in combination with the dual luciferase reporter constructs with the 2nd cistron controlled by either no IRES, the HCV IRES, a pestiviral IRES (BVDV), or a picornaviral IRES (EMCV). For the No-IRES construct (control) the results were normalized to the HCV IRES operating in the presence of GFP for all other constructs the results are graphed comparing IRES activity in the presence of NS5A to that in GFP. Cell lysates were collected 48 h post-transfection and subjected to dual luciferase assays as describe above. (C)NS5A from Replicon 4 does not up-regulate HCV IRES. GFP, 1BN-SI NS5A, CsA-1s NS5A and each individual Replicon 4 NS5A mutation (E260G, V284A, F288S, L307Q, R360G, and V448A) were separately transfected into Huh7 cells in combination with the dual-luciferase reporter construct containing the HCV IRES as described above. The activity of HCV IRES in GFP transfected cells was arbitrarily taken as 100% with the activities of the IRES under the different NS5A species calculated relative to this. All experiments were performed in triplicate and the mean values are graphed.

The foregoing description and examples have been set forth merely to illustrate the invention and are not intended to be limiting. Since modifications of the disclosed embodiments incorporating the spirit and substance of the invention may occur to persons skilled in the art, the invention should be construed broadly to include all variations and equivalents falling within the scope of the appended claims and equivalents thereof. All references cited hereinabove and/or listed below are hereby expressly incorporated by reference.

### REFERENCES

Forman, L. M., et al., The association between hepatitis C infection and survival after orthotopic liver transplantation. Gastroenterology, 2002. 122(4): p. 889-96.
2. Berenguer, M., Natural history of recurrent hepatitis C. Liver Transpl, 2002. 8(10 Suppl 1): p. S14-8.
3. Inoue, K., et al., Combined interferon alpha2b and cyclosporin A in the treatment of chronic hepatitis C: controlled trial. J Gastroenterol, 2003. 38(6): p. 567-72.
4. Levy, G., et al., Results of lis2t, a multicenter, randomized study comparing cyclosporine microemulsion with C2 monitoring and tacrolimus with C0 monitoring in de novo liver transplantation. Transplantation, 2004. 77(11): p. 1632-8.
5. Lauer, G. M. and B. D. Walker, Hepatitis C virus infection. N Engl J Med, 2001. 345(1): p. 41-52.
6. El-Serag, H. B. and A. C. Mason, Rising Incidence of Heaptocellular Carcinoma in the United States. N Engl J Med, 1999. 340(10): p. 745.
7. Treml, K., et al., Mixed Progress in Cancer Control in Wisconsin. Surveillance Brief, Wisconsin's Comprehensive Cancer Control Program, 2005. 1(1).
8. Lindenbach, B. D., et al., Complete Replication of Hepatitis C Virus in Cell Culture. Science, 2005.
9. Zhong, J., et al., Robust hepatitis C virus infection in vitro. Proc Natl Acad Sci USA, 2005.
10. Blight, K. J., A. A. Kolykhalov, and C. M. Rice, Efficient initiation of HCV RNA replication in cell culture. Science, 2000. 290(5498): p. 1972-4.
11. Ikeda, M., et al., Selectable subgenomic and genome-length dicistronic RNAs derived from an infectious molecular clone of the HCV-N strain of hepatitis C virus replicate efficiently in cultured Huh7 cells. J Virol, 2002. 76(6): p. 2997-3006.
12. Nakagawa, M., et al., Suppression of hepatitis C virus replication by cyclosporin a is mediated by blockade of cyclophilins. Gastroenterology, 2005. 129(3): p. 1031-41.
13. Watashi, K., et al., Cyclophilin B is a functional regulator of hepatitis C virus RNA polymerase. Mol Cell, 2005. 19(1): p. 111-22.
14. Everson, G. T., Impact of immunosuppressive therapy on recurrence of hepatitis C. Liver Transpl, 2002. 8(10 Suppl 1): p. S19-27.
15. Stangl, J. R., et al., Effect of antimetabolite immunosuppressants on Hepatitis C Virus. Transplantation, 2004. 77(4): p. 562-567.
16. Zekry, A., et al., A prospective cross-over study comparing the effect of mycophenolate versus azathioprine on allograft function and viral load in liver transplant recipients with recurrent chronic HCV infection. Liver Transpl, 2004. 10(1): p. 52-7.
17. Samonakis, D. N., et al., Immunosuppression and donor age with respect to severity of HCV recurrence after liver transplantation. Liver Transpl, 2005. 11(4): p. 386-95.
18. Yi, M., F. Bodola, and S. M. Lemon, Subgenomic hepatitis C virus replicons inducing expression of a secreted enzymatic reporter protein. Virology, 2002. 304(2): p. 197-210.
19. Shirota, Y., et al., Hepatitis C virus (HCV) NS5A binds RNA-dependent RNA polymerase (RdRP) NS5B and modulates RNA-dependent RNA polymerase activity. J Biol Chem, 2002. 277(13): p. 11149-55.
20. Enomoto, N., et al., Mutations in the nonstructural protein 5A gene and response to interferon in patients with chronic hepatitis C virus 1b infection. N Engl J Med, 1996. 334(2): p. 77-81.
21. Young, K. C., et al., Identification of a ribavirin-resistant NS5B mutation of hepatitis C virus during ribavirin monotherapy. Hepatology, 2003. 38(4): p. 869-78.
22. Ludmerer, S. W., et al., Replication fitness and NS5B drug sensitivity of diverse hepatitis C virus isolates characterized by using a transient replication assay. Antimicrob Agents Chemother, 2005. 49(5): p. 2059-69.
23. Kuiken, C., et al., The Los Alamos hepatitis C sequence database. Bioinformatics, 2005. 21(3): p. 379-84.
24. Yang, Z. and R. Nielsen, Estimating synonymous and nonsynonymous substitution rates under realistic evolutionary models. Mol Biol Evol, 2000. 17(1): p. 32-43.

We claim:

1. A method for determining susceptibility of a hepatitis C virus (HCV) in a sample to an immunosuppressant, wherein HCV genome comprises an NS5A and NS5B region, the method comprising determining the nucleic acid sequence of HCV, and comparing the viral nucleic acid sequence to that of a wild-type, CsA susceptible strain, wherein the existence of at least one mutation in the viral genome is indicative that the virus is resistant to CsA.

2. The method of claim 1, wherein the immunosuppressant is cyclosporine A (CsA).

3. The method according to claim 1, wherein the at least one mutation is one shown in Table 1.

4. The method according to claim 1, wherein the existence of at least two mutations is indicative that the virus is resistant to CsA.

5. The method according to claim 4, wherein the existence of at least one mutation in the NS5A region, and at least one mutation in the NS5B region is indicative that the virus is resistant to CsA.

6. The method according to claim 3, wherein the existence of all mutations identified in each of Replicons 2, 3, 4, 5, 4-5A, 4-5B, 6, 7, 8, 9, 10, 11, 12, 13 or 14 in Table 1 is indicative that the virus is CsA resistant.

7. The method according to claim 3, wherein the existence of all mutations identified in
each of Replicons 4, 5, 4-5A, 4-5B, 10, 12 in Table 1 is indicative that the virus is CsA resistant.

8. The method according to claim 3, wherein the existence of all mutations identified in Replicons 4-5A or 4-5B is indicative that the virus is CsA resistant.

9. The method according to claim 1, wherein the sample is a clinical sample obtained from a HCV infected patient.

10. The method according to claim 9, wherein the patient is liver-transplant patient.

11. The method according to claim 1, wherein the nucleic acid sequence is determined using a RT-PCR based technique.

12. The method according to claim 1, wherein nucleic sequence of regions of the viral genome encoding viral proteins NS5A or NS5B or both is determined.

13. An isolated polynucleotide molecule comprising at least 15 contiguous nucleotides shown in FIG. 4, and at least one mutation identified in Table 1.

14. A gene chip comprising at least two isolated polynucleotide molecules according to claim 13.

15. A kit comprising at least one isolated polynucleotide of claim 13, and a means for determining whether a sample contains a nucleic acid molecule that comprises the nucleotide sequence of the polynucleotide.

16. The kit according to claim 15, wherein the means comprises reagents suitable for a PCR or a hybridization reaction that utilizes the polynucleotide molecule as a primer or a probe.

17. A method of monitoring the development CsA resistance in a HCV patient, the method comprising determining the nucleic acid sequence of the HCV in a sample from the patient, and determining the susceptibility of the HCV to CsA according to claim 2, wherein the appearance of a mutation identified in Table 1A or 1B is indicative that the HCV has developed CsA resistance.

18. The method according to claim 17, wherein the patient is a liver transplant patient afflicted by HCV infection

19. A method for managing HCV treatment in a liver-transplant patient, the method comprising determining whether the HCV in the patient is CsA resistant according to claim 2, and administering to the patient a suitable immunosuppressant or antiviral drug or both accordingly.

20. The method according to claim 19, wherein the antiviral drug is interferon.

21. A CsA-resistant HCV replicon, comprising the nucleotide sequence of the isolated polynucleotide molecule according to claim 13.

22. The replicon of claim 17, which comprises a sequence is selected from the group consisting of the nucleotide sequence of Amplicon 2, 3, 4, 5, 4-5A, 4-5B, 6, 7, 8, 9, 10, 11, 12, 13 and 14 in Table 1.

23. A method for screening for anti-viral pharmaceutical compounds, the method comprising applying a candidate compound to a cell culture that comprises a CsA resistant replicon according to claim 21, and determining whether the candidate compound inhibits viral replication or viral protein synthesis, wherein a candidate that shows inhibitory effects is an antiviral compound.

24. The method according to claim 23, wherein the candidate compound is a RNA polymerase inhibitor.

25. The method according to claim 24, wherein the polymerase inhibitor is a nonnucleoside.

26. A method for treating a patient infected with HCV, the method comprises (1) treating the patient CsA for a period of time, (2) obtaining a sample from the patentient containing HCV and determining CsA susceptibility of the HCV in the sample according to claim 1, and (3) treating patient with interferon if the HCV is found to be CsA resistant.

27. The method according to claim 26, wherein the HCV is found to contain at least one mutation in the region of aa 237 to aa 306.

28. The method according to claim 27, wherein the HCV is found to contain one mutation at position F288.


SEQUENCE LISTING (1 sequence)

SEQ ID NO: 1 - DNA, 2488 bp, Human Hepatitis C Virus
gctaagcgtaggttggccagggggtctcccccctccttggccagctcttcagctatccagctgtctgcgccctccttgagggcgacatgcactacccattcttcctataatcttgactctccggacgtcgacctcattgaggccaacctcctgtggcggcaggagatgggcggaaacatcacccgcgtggagtcggagaacaaggtggtagtcctagactctttcgagccgcttcgagcggagggggatgagaatgaaatatccattgcggcggagatcctgcggaagtccaagaagttccccgcggcgatacccatatgggcacggccggattacaatcctccattgttagagtcttggaagaacccggactacgtccctccggtggtacacgggtgcccattgccacctgtcaaggcccctccaataccacctccacggagaaaaaggacggttgtcctgacggactccaccgtgtcttctgttttggcggagctcgctaccaaaaccttcggcagctccgaattgtcggccgccgacagcggcacggcgaccgcccctcctgaccagacctccgacaacggcggcaaagactccgacgctgagtcatgctcctctatgcccccccttgagggggagccgggggaccccgatctcagcgacgggtcttggtctaccgtgagcgaggaggctggtgagagcgtcgtctgctgctcaatgtcctacacatggacaggtgccctgatcacgccatgcgccgcggaagaaagcaagctgcccatcaacgcgttgagcaactctttgctgcgccatcacaacatggtctacgccacgacatcccgcagcgcgggcctgcggcagaagaaggtcacctttgacagactgcaggtcctggatgaccattaccgggacgtgcttaaggagatgaaggcaaaggcgtccacagtcaaggctaaacttctatccatagaagaagcctgccgcctgacgcccccacattcggccaaatccaagtttggctatggggcaaaggacgtccggaacctatccagcagggccatcaaccacatccgctccgtgtgggaggacttgctggaggacactgtgacaccaattgacaccaccgtcatggcaaagaatgaggttttctgcgtccaaccagagaagggaggccgcaagccagcccgccttatcgtattcccagatttgggagttcgtgtatgcgagaagatggctctctacgatgtggtctccacccttcctcaagccgtgatgggctcctcatacggattccagtactctcccgggcagcgggtcgagttcctggtaaaagcctggaaatcaaagaaaaaccctatgggcttctcatatgacacccgctgttttgactcaacggtcactgagaatgacatccgtgttgaggagtcaatttaccaatgttgtgacttggcccccgaagccagacaggctataaaatcgctcacagagcggctttatatcgggggtcccctgactaattcaaaagggcagagctgtggttatcgccggtgccgcgcgagcggcgtgctgacgactagctgcggtaataccctcacatgttacttgaaagcctctgccgcctgtcgagctgcaaagctccaggactgcacgatgctcgtgaacggggacgaccttgtcgttatctgcgaaagcgcgggaacccaggaggatgcggcgagcctacgagtcttcacggaggctatgactaggtactccgccccccccggggacttgccccaaccagaatacgacttggagttgataacatcatgttcctccaatgtgtcggtcgcgcacgatgcatctggcaaaagggtgtactacctcactcgcgatcccaccacccccatcgcacgggctgcgtgggaaacagctagacacactccagttaactcctggctaggcaacattatcatgtatgcgcccaccttatgggcaaggatgattctgatgacccatttcttctccatccttctagctcaggagcaacttgaaaaagccctggattgccaaatctacggggcctgttactccattgagccacttgacctacctcagatcattgaacgactccatggtcttagcgcattttcactccatagttactctccaggtgagatcaatagggtggcttcatgcctcaggaaacttggggtaccgcccttgcgagtctggagacatcgggccaggagcgtccgcgctaaactactgtcccagggggggagggccgccacttgcggcaaatacctcttcaactgggcagtaaagaccaagctcaaactcactccaatcccggctgcgtcccagttggacttatccggctggttcgttgctggctacagcgggggagacatatatcacagcctgtctcgtgcccgaccccgctggttcatgctgtgcctactcctactttctgtaggggtaggcatctacttgctccccaatcgat